(19) United States
(12) Patent Application Publication
(10) Pub. No.: US 2016/0095604 A1
(43) Pub. Date: Apr. 7, 2016

(54) Mixing Syringe

(71) Applicant: Daniel Gelbart, Vancouver (CA)

(72) Inventor: Daniel Gelbart, Vancouver (CA)

(21) Appl. No.: 14/869,783

(22) Filed: Sep. 29, 2015

Related U.S. Application Data
(60) Provisional application No. 62/058,101, filed on Oct. 1, 2014.

(51) Int. Cl. A61B 17/12; A61M 5/24; A61M 5/28

(52) U.S. Cl. CPC A61B 17/12186; A61M 5/284; A61M 5/2448

(57) ABSTRACT

A perforated separator compartment or liquid permeable bag is attached to the input port in a regular syringe. The perforated compartment only allows liquids to exit the compartment into the main chamber of the syringe, restricting solid particles to the near proximity of the input port of the syringe. When the plunger of the syringe is pressed, the saline solution emerges from holes into the compartment, stirring up the settled particles. As the ejection continues, the compartment is compressed towards the input port by the plunger in order to eliminate any unused volume.

### FIELD OF THE INVENTION

The invention is in the medical field, and is particularly useful in percutaneous procedures such as embolization.

### BACKGROUND OF THE INVENTION

In certain medical procedures, such as blood vessel embolization, it is desired to inject particles into the body. The procedure is a minimally invasive alternative to surgery. The purpose of embolization is to prevent blood flow to an area of the body, which effectively can shrink a fibroid, such as a uterine fibroid. It can also shrink a tumour or block an aneurysm. It is typically done by injecting blocking particles into a blood vessel.

The procedure is carried out as an endovascular procedure by a radiologist in an interventional suite. It is common for most patients to have the treatment carried out with little or no sedation, although this depends largely on the organ to be embolized.

Access to the organ in question is acquired by means of a guidewire and catheter. The position of the correct artery or vein supplying the undesired tissue in question is located by X-Ray images. These images are then used as a map for the radiologist to gain access to the correct vessel by selecting an appropriate catheter and or wire, depending on the shape of the surrounding anatomy.

The blocking particles are mixed into a saline solution, sometimes a contrast agent is added (to make the solution opaque to X-Rays). The blocking particles have to be of certain sizes, typically between 0.1 mm to 1 mm, in order to block the blood vessel at the right diameter. Such particles tend to settle very quickly out of the solution as they are heavier than water, causing an uneven concentration of particles during the injection. The settling occurs in as little as a few seconds. It is inconvenient to keep shaking the syringe used for injection, as the whole process is performed in a few seconds and the doctor has to concentrate on injecting the correct amount. It is desired to have a syringe that can keep the particles uniformly dispersed in the saline solution regardless of delays in the injection process or speed of the injection. Since the syringes used are low cost disposable items, it is desired that the device used to keep the particles uniformly dispersed will also be very low cost and disposable. The ideal mixing syringe needs the following attributes:

A. Ability to be re-filled multiple times during a procedure. This rules out any single-use designs, typically using the rupturing of a membrane to allow mixing.

B. Generate a strong mixing action, preferable by creating a vortex in the mixture.

C. Use the minimum modification to a standard syringe.

Prior art mixing syringes, such as disclosed in U.S. Pat. No. 7,883,490 are designed to mix together two materials stored separately in two compartments. They are not designed to stir up a pre-mixed solution. Prior art syringes designed to stir-up embolization mixtures, such as disclosed in US2009/0247985, are needlessly complex. Also, many of the prior art mixing syringes are not designed to be filled with the pre-mixed solution just before use. This is required during embolization, as the correct volume and ratio of saline, particles and contrast agent has to be customized to the procedure by the doctor. The current invention acts as a regular syringe, allowing filling and injecting at any time, but it keeps the solution stirred up during injection. Similar to a regular syringe, it can be re-used several times during a procedure, if more particles have to be injected. The invention can be manufactured out of a regular syringe, which is a very low cost item.

### SUMMARY OF THE INVENTION

A perforated separator compartment or liquid permeable bag is attached to the input port in a regular syringe. The perforated compartment only allows liquids to exit the compartment into the main chamber of the syringe, restricting solid particles to the near proximity of the input port of the syringe. When the plunger of the syringe is pressed, the saline solution emerges from holes into the compartment, stirring up the settled particles. As the ejection continues, the compartment is compressed towards the input port by the plunger in order to eliminate any unused volume.

### DESCRIPTION OF THE FIGURES

FIG. 1 shows the preferred embodiment of the invention.

FIG. 2 shows the input port of the preferred embodiment of the invention.

FIG. 3 depicts the preferred embodiment of the invention during the filling cycle.

FIG. 4 depicts the preferred embodiment of the invention during the ejection cycle.

FIG. 5 depicts an alternative embodiment of the invention during the ejection cycle.

### PREFERRED EMBODIMENT

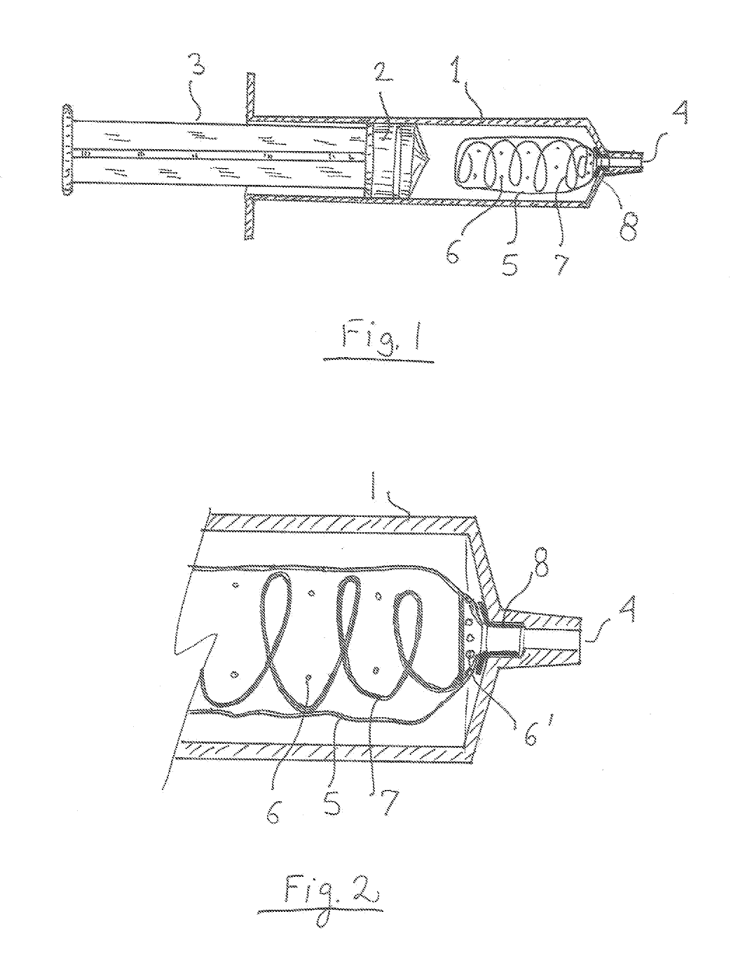

Referring to FIG. 1 a standard disposable syringe typically comprises of a body 1, plunger 3 incorporating seal 2 and an output port 4, typically a tapered Luer fitting. A perforated bag or compartment 5 is added inside body 1. The bag is connected to the output port either directly (e.g. by adhesive bonding) or held in place by insert 8. A structural reinforcement feature 7 prevents the bag from collapsing. The preferred embodiment of this device uses a spring as a structural reinforcement feature. Spring 7 can be replaced by a reinforcing structure in the bag wall such as corrugations, turning the bag into a bellows. The bag is perforated in several places with holes or slots 6 smaller than the particles the bag contains. To make syringe work at low feed rates, a small number of small diameter perforations are used, to achieve reasonable jet velocity. When the syringe is filled with a mixture of particles in a liquid, typically saline solution, the mixture is sucked in via port 4, however the particles are trapped inside the bag 5. The liquid can pass via holes 6 and fill the rest of the syringe. One purpose of bag 5 is to ensure the particles remain in close proximity to the syringe port 4, allowing them to easily mix with the liquid component of the suspension during the ejection phase of the cycle. If the particles settle to the bottom of the bag 5, they will still mix with the ejecting liquid due to the proximity and strength of the fluid's current. This is due to the increase in flow and turbulence of the liquid as it passes through holes 6. When describing the device, we refer to the proximal region as the end where the plunger 3 enters the syringe body 1 and the distal region as the end towards the ejection port 4.

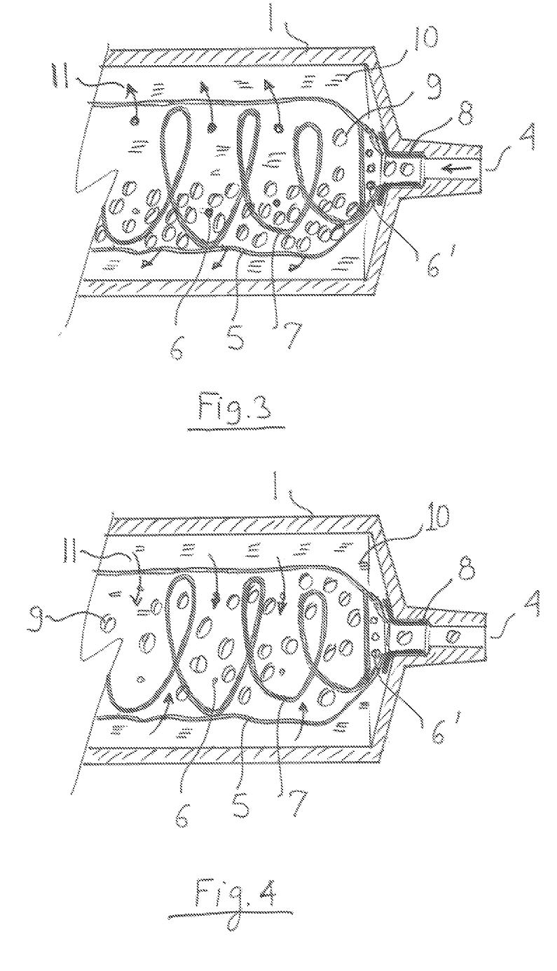

Details of the region the bag 5 connects to the syringe are shown in FIG. 2. Besides a plurality of holes 6 there are extra holes 6′ near the point the bag is attached to the syringe. The function of holes 6′ is to allow any air bubbles in the volume outside the bag to escape when syringe is held vertically. Holes 6′ can be the same size as holes 6 or larger. Sometimes it is advantageous to replace the holes with narrow slots or cuts, typically 50 um wide and about 1 mm long. The advantage of slots is that even small particles cannot get through and there is less likelihood of plugging by large particles. The bag can be made from any flexible material, such as polyethylene, and can be molded or made from a folded and heat sealed sheet. Typical thickness of bag material is 0.03 to 0.1 mm.

Referring now to FIG. 3, during the filling cycle the mixture enters via port 4 but particles 9 which are larger than holes or slots 6 are trapped inside bag 5. The whole inside volume of the syringe fills with liquid 10, including the volume of bag 5. Arrows 11 show the direction of liquid flow through holes 6. After a short time the particles settle at the bottom of the bag 5 as shown. The settling time depends on particle size and density, but typically settling become visible after as little as one second.

FIG. 4 shows the ejection of particles from the syringe. Inward movement of the plunger (not shown) forces liquid 10 via holes or slots 6 as shown by arrows 11. Since the holes are small, typically 0.05-0.5 mm, multiple fine jets of liquid are created, stirring up the mixture and preventing the particles from settling as long as plunger is moving. This creates a uniform mixture of liquid and particles at port 4. The bag 5 is compressed during the ejection cycle, to allow all or some of the mixture to exit through port 4.

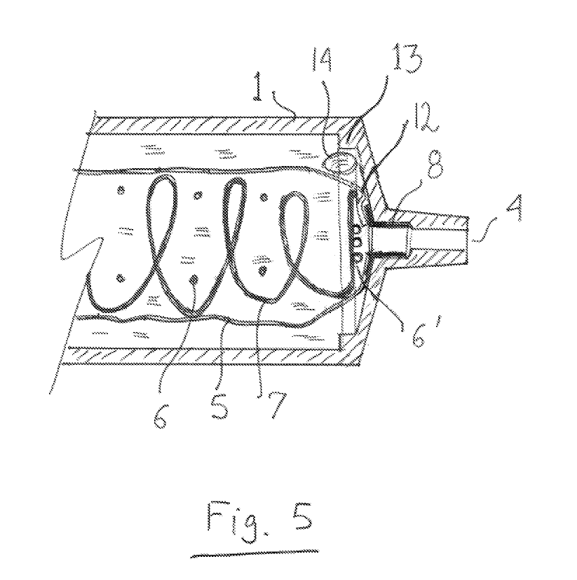

Referring now to FIG. 5, several method can be used to allow trapped air bubbles (created during filling) to escape easily. The air bubbles which are created inside the bag escape easily as they do in a regular syringe. The bubbles 14 outside the bag can escape via holes 6′ when syringe is held vertically with port 4 pointing up. When holes 6′ are made large, to allow easy air escape, it may be desirable to add a valve that exposes holes 6′ only during part of the ejection cycle. One such valve can be created by allowing the spring 7 to fold the bag over the area holes 6′ are located. As long as the spring 7 is compressed holes 6′ will be sealed as fold 12 in bag 5 forms a ring seal. At the end of the filling cycle the spring is no longer compressed, as seen in FIG. 1. When syringe is now held vertically, air bubble 14 can escape via holes 6′ until spring is compressed by plunger. Since holes 6′ are blocked during most of the filling and ejection cycle, they can be made large, even larger than the particles. The occasional escape of a particle through these holes is not a problem. Other valve mechanism that expose air escape holes for part of the plunger travel can be made using flaps, side ports in body 1 and other valve styles.

An alternative to letting all air outside bag 5 escape is to prevent the air from entering the bag and thus reaching port 4. A simple way to prevent air bubble 14 from being compressed and forced to enter bag 5 is to provide a stop 13, not allowing the plunger from reaching the end of syringe body 1. In this case holes 6′ can be omitted. Note that at the very end of the ejection cycle there are no more particles in bag 5 and stopping the plunger just before the end does not waste particles.

We use the term suspension throughout this specification, which is typically defined as a mixture with a liquid component and solid component, where the solid component can settle or separate from the mixture. It should be noted that any mixture with particles or a component that can be restricted by the perforated bag can be used with the disclosed invention.

We use the term bag to describe the compartment that restricts movement of the suspended particles, but this can be any structure that is permeable to the carrier liquid, but not the solid particles. For example, the particle restricting compartment could be a fine mesh. Similarly, we use the term perforated to mean any hole that allows liquid to pass through the compartment wall, but restricts the movement of the solid particles. In the example used above, perforation could refer to the space between the fibres in the mesh material.

1. A syringe comprising a plunger, a perforated bag and a port, wherein perforated bag restricts movement of solid particles when the syringe is filled with a mixture of liquid and said solid particles, the plunger ejects the suspension of liquid and solid particles from the syringe and the bag forces the liquid to mix with said solid particles.

2. A syringe as in 1, wherein said bag comprises a reinforcement structure.

3. A syringe as in 1, wherein said bag comprises perforations that are smaller than said solid particles.

4. A syringe as in 1, wherein said bag comprises a proximal end and said proximal end of the bag is compressed by a plunger during ejection.

5. A syringe as in 1, wherein said bag comprises a distal end and the distal end is attached to the port.

6. A syringe as in 1, wherein said bag comprises a distal end and the plunger ejects said mixture through the port and said plunger is blocked from reaching the distal end of said syringe.

7. A syringe as in 1, wherein said syringe includes a vent for releasing trapped air from around the bag and said vent closes when the plunger starts moving.

8. A method of mixing a liquid with solid particles that comprises the following steps:
filling a syringe that comprises a perforated bag attached to port of said syringe,
wherein said bag retains said solid particles during filling of syringe and said bag does not block movement of liquid during filling of syringe,
ejecting the mixture from the syringe with a plunger, wherein said liquid goes through perforations in said bag and the liquid mixes with the solid particles in said bag before ejecting through said port.

9. A method as in 8, wherein said bag comprises a reinforcement structure.

10. A method as in 8, wherein said bag comprises perforations that are smaller than said solid particles.

11. A method as in 8, wherein said bag comprises a proximal end and said proximal end of the bag is compressed by a plunger during ejection cycle.

12. A method as in 8, wherein said bag comprises a distal end and the distal end is adhered to the port.

13. A method as in 8, wherein said plunger ejects said mixture through the port and the plunger is blocked from reaching the distal end of said syringe.

14. A method as in 8, wherein said bag comprises a distal end, wherein said distal end comprises slots configured to eject trapped air from said syringe, wherein the slots are narrower than the solid particles.

15. A method as in 8, wherein said syringe includes a vent for releasing trapped air from around the bag and said vent closes when the plunger starts moving.

16. A method of mixing liquid and solid particles in a syringe by increasing turbulence inside said syringe during ejection of said mixture, wherein the turbulence is increased by forcing the liquid through holes and said holes are smaller than the solid particles.

17. A method as in 15, wherein said syringe comprises a foldable bag, wherein said bag comprises holes to increase the turbulence of said mixture and a reinforcement structure.

18. A method as in 15, wherein said mixture is ejected from the syringe with a plunger and said plunger is blocked from reaching the distal end of the syringe.

19. A method as in 15, wherein the syringe comprises a foldable bag, said bag is configured to vent air through slots in the distal end of the bag and said slots are narrower than the solid particles.

20. A method as in 15, wherein said syringe comprises a foldable bag, said bag includes a vent for releasing trapped air from around the bag and the vent closes when the mixture starts ejecting from the syringe.
